SEQUENCE LISTING (7 sequences)

SEQ ID NO: 1 - DNA, 858 bp, Homo sapiens
  CDS (1)..(858): Coding sequence for variant human dimethylarginine dimethylaminohydrolase 1 (DDAH1) protein
atggccgggctcggccaccccgccgccttcggccgggccacccacgccgtggtgcgggcgctacccgagtcgctcggccagcacgcgctgagaagcgccaagggcgaggaggtggacgtcgcccgcgcggaacggcagcaccagctctacgtgggcgtgctgggcagcaagctggggctgcaggtggtggagctgccggccgacgagagccttccggactgcgtcttcgtggaggacgtggccgtggtgtgcgaggagatggccctcatcacccgacccggggcgccgagccggaggaaggaggttgacatgatgaaagaagcattagaaaaacttcagctcaatatagtagagatgaaagatgaaaatgcaactttagatggcggagatgttttattcacaggcagagaattttttgtgggcctttccaaaaggacaaatcaacgaggtgctgaaatcttggctgatacttttaaggactatgcagtctccacagtgccagtggcagatgggttgcatttgaagagtttctgcagcatggctgggcctaacctgatcgcaattgggtctagtgaatctgcacagaaggcccttaagatcatgcaacagatgagtgaccaccgctacgacaaactcactgtgcctgatgacatagcagcaaactgtatatatctaaatatccccaacaaagggcacgtcttgctgcaccgaaccccggaagagtatccagaaagtgcaaaggtttatgagaaactgaaggaccatatgctgatccccgtgagcatgtctgaactggaaaaggtggatgggctgctcacctgctgctcagttttaattaacaagaaagtagactcctga

SEQ ID NO: 2 - protein, 285 aa, Homo sapiens
MAGLGHPAAFGRATHAVVRALPESLGQHALRSAKGEEVDVARAERQHQLYVGVLGSKLGLQVVELPADESLPDCVFVEDVAVVCEEMALITRPGAPSRRKEVDMMKEALEKLQLNIVEMKDENATLDGGDVLFTGREFFVGLSKRTNQRGAEILADTFKDYAVSTVPVADGLHLKSFCSMAGPNLIAIGSSESAQKALKIMQQMSDHRYDKLTVPDDIAANCIYLNIPNKGHVLLHRTPEEYPESAKVYEKLKDHMLIPVSMSELEKVDGLLTCCSVLINKKVDS

SEQ ID NO: 3 - DNA, 858 bp, Homo sapiens
  CDS (1)..(858): Coding sequence for human dimethylarginine dimethylaminohydrolase 1 (DDAH1) protein
atggccgggctcggccaccccgccgccttcggccgggccacccacgccgtggtgcgggcgctacccgagtcgctcggccagcacgcgctgagaagcgccaagggcgaggaggtggacgtcgcccgcgcggaacggcagcaccagctctacgtgggcgtgctgggcagcaagctggggctgcaggtggtggagctgccggccgacgagagccttccggactgcgtcttcgtggaggacgtggccgtggtgtgcgaggagacggccctcatcacccgacccggggcgccgagccggaggaaggaggttgacatgatgaaagaagcattagaaaaacttcagctcaatatagtagagatgaaagatgaaaatgcaactttagatggcggagatgttttattcacaggcagagaattttttgtgggcctttccaaaaggacaaatcaacgaggtgctgaaatcttggctgatacttttaaggactatgcagtctccacagtgccagtggcagatgggttgcatttgaagagtttctgcagcatggctgggcctaacctgatcgcaattgggtctagtgaatctgcacagaaggcccttaagatcatgcaacagatgagtgaccaccgctacgacaaactcactgtgcctgatgacatagcagcaaactgtatatatctaaatatccccaacaaagggcacgtcttgctgcaccgaaccccggaagagtatccagaaagtgcaaaggtttatgagaaactgaaggaccatatgctgatccccgtgagcatgtctgaactggaaaaggtggatgggctgctcacctgctgctcagttttaattaacaagaaagtagactcctga

SEQ ID NO: 4 - protein, 285 aa, Homo sapiens
MAGLGHPAAFGRATHAVVRALPESLGQHALRSAKGEEVDVARAERQHQLYVGVLGSKLGLQVVELPADESLPDCVFVEDVAVVCEETALITRPGAPSRRKEVDMMKEALEKLQLNIVEMKDENATLDGGDVLFTGREFFVGLSKRTNQRGAEILADTFKDYAVSTVPVADGLHLKSFCSMAGPNLIAIGSSESAQKALKIMQQMSDHRYDKLTVPDDIAANCIYLNIPNKGHVLLHRTPEEYPESAKVYEKLKDHMLIPVSMSELEKVDGLLTCCSVLINKKVDS

SEQ ID NO: 5 - DNA, 21 bp, Artificial Sequence
  primer directed to the amplification of human dimethylarginine dimethylaminohydroase 1 (DDAH1) exon
gtcccccgcctccgcatactt

SEQ ID NO: 6 - DNA, 22 bp, Artificial Sequence
  primer directed to the amplification of human dimethylarginine dimethylaminohydroase 1 (DDAH1) exon
ccacctgcccgagaccgtacaa

SEQ ID NO: 7 - DNA, 18 bp, Artificial Sequence
  primer directed to the sequencing of human dimethylarginine dimethylaminohydroase 1 (DDAH1) exon
ccgacgggaagttgtgaa

(19) United States
(12) Patent Application Publication
(10) Pub. No.: US 2004/0214264 A1
(43) Pub. Date: Oct. 28, 2004

(54) Nucleic acid molecule encoding a variant DDAH 1 protein and uses thereof

(76) Inventors: Veli-Pekka Valkonen, Kuopio (FI); Jukka T. Salonen, Jannevirta (FI); Mia Pirskanen, Kuopio (FI); Tomi-Pekka Tuomainen, Kuopio (FI); Juha Laakso, Helsinki (FI); Reijo Laaksonen, Monninkyla (FI)

Correspondence Address: BIRCH STEWART KOLASCH & BIRCH, PO BOX 747, FALLS CHURCH, VA, 22040-0747, US

(21) Appl. No.: 10/125,456

(22) Filed: Apr. 19, 2002

(51) Int. Cl. C12Q 1/68; C07H 21/04; C12N 9/80; C12P 21/02; C12N 5/06

(52) U.S. Cl. 435/69.1; 435/320.1; 435/325; 536/23.2; 435/228; 435/6

(57) ABSTRACT

This invention relates to a nucleic acid encoding a variant human DDAH protein and to said variant DDAH protein as well as a method for screening a subject to determine if said subject is a carrier of a variant gene that encodes said variant DDAH protein. Further this invention relates to a method for detecting or diagnosing a risk of, or predisposition to, cardiovascular disease and diabetes in a subject, for selecting treatment in a subject and for selecting subjects for studies testing cardiovascular and diabetes drugs, a method for the treatment of type 2 diabetes as well as to transgenic animals.

### FIELD OF THE INVENTION

This invention relates to a nucleic acid sequence encoding a variant human dimethylarginine dimethylaminohydrolase (DDAH 1) protein and to said variant DDAH 1 protein as well as a method for screening a subject to determine if said subject is a carrier of a variant gene that encodes said variant DDAH protein. Further, this invention relates to a method for detecting or diagnosing a risk of, or predisposition to, cardiovascular diseases and diabetes in a subject, a method for targeting treatment in a subject, and a method for selecting subjects for studies testing antidiabetic agents, as well as a method for the treatment of type 2 diabetes.

### BACKGROUND OF THE INVENTION

The publications and other materials used herein to illuminate the background of the invention, and in particular, to provide additional details with respect to its practice, are incorporated by reference.

Nitric oxide is a gaseous mediator synthesized from amino acid L-argine by the action of nitric-oxide synthases. This enzyme has three isoforms (NOS 1-3) mainly acting in endothelial, neuronal, and inducible circumstances. In the cardiovascular system endothelium has an important role in monocyte and leucosyte adhesion, platelet aggregation, thrombosis, smooth-muscle cell proliferation, and vasoregulation.

Endothelial dysfunction defined as the impaired ability of vascular endothelium to stimulate vasodilation plays a key role in the development of atherosclerosis and in various pathological conditions which predispose to atherosclerosis, such as hypercholesterolemia, hypertension, type 1 and 2 diabetes, hyperhomocyst(e)inemia, chronic renal failure, obesity (abdominal), infection, aging, smoking and hard physical activity. A major cause of the endothelial dysfunction is decreased bioavailability of nitric oxide (NO), a potent biological vasodilator produced in vascular endothelium from L-arginine by the endothelial NO synthase (eNOS).

ADMA is a naturally occurring compound that inhibits nitric oxide synthases. It is formed by methylation of arginine residues in proteins and is metabolised by the enzyme dimethylarginine dimethylaminohydrolase (DDAH). Concentrations of ADMA are raised in young hypercholesterolaemic patients (Boger et al 1998), correlate with intima media thickness in healthy middle-aged individuals (Miyazaki et al 1999) and in patients with type 2 diabetes (Fard et al 2000). ADMA has shown to be a predictor of acute coronary events among middle-aged men (Valkonen et al 2001).

Hyperglycaemia is the most typical phenomenon in diabetes. The presence of advanced glycation end-products (AGE) is closely related to hyperglycaemia and their patho-biochemistry explains many of the changes in diabetes related complications. The association between AGE and ADMA has not been studied earlier.

DDAH1 gene is located in chromosome 1. This chromosome has been implicated in susceptibility to several diseases, including familial combined hyperlipidemia, premature coronary artery disease (CAD), non-insulin-dependent diabetes mellitus and diastolic hypertension. Also concentrations of high-density lipoprotein (HDL) cholesterol have shown suggestive linkage to chromosome 1. Impaired endothelial function and the altered production of nitric oxide have been implicated in the pathology of all those diseases.

The sequence homology between human DDAH 1 and mouse or rat DDAH 1 is very high. Thus there are several conserved regions in the gene coding the DDAH 1. There are totally three longer amino acid sequences that are identical between human and the mouse and rat: amino acids 87^{th} to 94^{th}, 173^{rd} to 177^{th} and 221^{st} to 228^{th}.

### SUMMARY OF THE INVENTION

The object of this invention is to provide a method for screening a subject to assess if an individual is at risk to develop cardiovascular disease and diabetes, based on the genotype of DDAH 1 gene and a method to target antihypertensive, antiischaemic and blood glucose lowering treatments. A further object of the invention is to provide a method for the selection of experimental animals and human subjects for studies testing antihypertensive, anti-ischaemic and antidiabetic effects of drugs. A third object of the invention is to provide a method for the treatment of type 2 diabetes in a human or animal. A fourth object of the invention is to provide a transgenic animal with a gene encoding human variant DDAH 1.

The present invention concerns a method for detecting a risk of cardiovascular disease and diabetes in a subject by determining the pattern of alleles encoding a variant DDAH, i.e. to determine if said subject's genotype of the human DDAH is variant type, comprising the steps of

a) providing a biological sample of the subject to be tested,

b) detecting the presence of variant genotype of the human DDAH 1 gene in the biological sample, the presence of variant genotype indicating an increased risk of cardiovascular diseases and diabetes in said subject.

According to the invention, the method allows for establishing whether said subject is of said variant genotype or not, a presence of said variant genotype in the biological sample, such as a blood sample or a buccal swab, thus indicating an increased risk of the subject to develop cardiovascular disease and diabetes, and/or indicating the subject being in need for treatment, such as DDAH agonist or other DDAH activity inducing therapy.

Preferably, the present invention concerns a method for detecting a risk of cardiovascular disease and diabetes in a subject by determining the pattern of alleles encoding a variant DDAH, i.e. to determine if said subject's genotype of the human DDAH is either Thr/Met or Met/Met type, comprising the steps of

a) providing a biological sample of the subject to be tested,

b) detecting the presence of Thr/Met or Met/Met genotype of the human DDAH 1 gene in the biological sample, the presence of Thr/Met or Met/Met genotype indicating an increased risk of cardiovascular diseases and diabetes in said subject.

According to the invention, the method allows for establishing whether said subject is of said Thr/Met or Met/Met genotype or not, a presence of said Thr/Met or Met/Met genotype in the biological sample, such as a blood sample or a buccal swab, thus indicating an increased risk of the subject to develop cardiovascular disease and diabetes, and/or indicating the subject being in need for treatment, such as DDAH agonist or other DDAH activity inducing therapy.

Said method can thus include a step of identifying a subject having an increased risk to develop cardiovascular disease and diabetes, and/or a subject in need of therapy, such as subtype-selective or nonselective DDAH agonist or antagonist therapy for cardiovascular disease and diabetes.

The invention also concerns a method as defined comprising a further step of

c) assessing at least one of the two following

i) the subject's risk to develop cardiovascular disease and diabetes, or

ii) the subject's need for subtype (1 vs. 2) selective or nonselective DDAH agonist therapy for cardiovascular disease and diabetes,

based on whether said subject is of said Thr/Met or Met/Met genotype or not.

A further object of the invention is a method for treating, or targeting the treatment of cardiovascular disease and diabetes in a subject by determining the pattern of alleles encoding a variant DDAH, i.e. by determining if said subject's genotype of the human DDAH is of the Thr/Met or Met/Met type, comprising the steps presented above, and treating a subject of the Thr/Met or Met/Met genotype with a drug affecting NOS and/or DDAH activity or with a supplementation affecting substrate for NO production.

The present invention is also directed to a kit for detecting a risk of cardiovascular disease and diabetes in a subject, comprising means for determining the pattern of alleles encoding a variant DDAH in a biological sample, as well as its use for detecting a risk of cardiovascular disease and diabetes.

The invention also provides a nucleic acid sequence comprising a nucleotide sequence encoding a variant DDAH protein with a substitution in the 87^{th} amino acid of the polypeptide. This place in the polypeptide is highly conserved area i.e. many other mammals have exactly the same amino acid sequence emphasizing the importance of the area.

It is plausible that any amino acid substitution in the same conserved region, i.e. from 87^{th} to 94^{th} amino acid or in another conserved region such as from the 173^{rd} to 177^{th} and 221^{st} to 228^{th} amino acid of the DDAH 1 protein will have the same role and effect on the disease risks and drug preference than that of the 87^{th} amino acid.

The invention further provides a variant DDAH 1 polypeptide.

### DETAILED DESCRIPTION OF THE INVENTION

The present invention relates to a nucleic acid molecule encoding a variant human DDAH 1, said variant DDAH protein and a method to assess the risk of individuals to develop cardiovascular disease and diabetes in mammals, a method for selecting treatment and selecting subjects testing treatments for cardiovascular disease and diabetes, a method for the treatment of type 2 diabetes in a human or animal as well as to transgenic animals with the variant DDAH gene.

The word treating shall also be understood to include preventing.

We have identified a variant form (SEQ ID NO: 1) of the human DDAH gene (SEQ ID NO: 3). This variant gene encodes a protein (SEQ ID NO: 2) with a substitution in the 87^{th} amino acid of the polypeptide.

The results to be presented below show that in a population sample of 1608 Finnish middle-aged men, carriers of the DDAH 1 mutation described above, thus representing a Thr/Met or Met/Met genotype of the DDAH 1 gene, have a significantly elevated risk for cardiovascular disease and diabetes. Based on these results and previous publications referred to above it can be postulated that this Thr/Met and Met/Met genotype is related to an impaired capacity to inactivate ADMA by metabolizing it to citrulline. Since altered DDAH function seems to be of relevance in the pathogenesis of cardiovascular disease and diabetes, we believe it could also be of relevance in subjects with the wildtype and the Thr/Met and Met/Met genotypes when other risk factors for cardiovascular disease and diabetes are present.

DDAHs regulate the metabolism of ADMA and nitric oxide, which are relevant in disorders such as cardiovascular disease and diabetes. These subjects will especially benefit from treatment with a DDAH enhancing therapies, and will be at increased risk for adverse effects if DDAH antagonists are administered to them. Therefore, a gene test recognizing subjects with a Thr/Met or Met/Met variant of the DDAH gene will be useful in diagnostics and patient selection for specific therapeutic procedures and clinical drug testing trials. A gene test recognizing the Thr/Met or Met/Met genotype of the DDAH is useful in assessing an individual's risk to develop cardiovascular disease and diabetes related to the Thr/Met or Met/Met genotype. The test can be used to set a specific subdiagnosis of cardiovascular disease and diabetes, based on its genetic etiology.

Furthermore, a gene test recognizing the Thr/Met or Met/Met genotype of the DDAH is useful in selecting drug therapy for patients with cardiovascular disease and diabetes. Such drugs belong to three main categories: 1) drugs stimulating NO generation, for example biogenic amines, some currently used cardiovascular drugs, such as dihyrdopyridines, statins, inhibitors of angiotensin-converting enzymes, angiotensin II receptor antagonists and some antidiabetic drugs, such as the biguanide family; 2) drugs dealing with spesific points in actions of endogenous NO, for example inhibitors of phosphodiesterase enzymes or using selective inhibitors of cGMP phosphodiesterase; and 3) drugs donating NO, i.e. NO replacement therapy using NO gas or the drugs that donate NO, for example organic nitrates, S-nitroso-glutathione and eNOS substrate, L-arginine, or its cofactor, tetrahydrobiopterin (BH4). Also, a new class of NO donor agents in which the class members are clinically used nonsteroidal antiinflammatory drugs which are jointly in preparation with a readily hydrolyxable nitrate ester moiety belong to this 3rd group. Also novel strategies, which may produce beneficial changes in the vascular endothelium, include the use of natural extracts from plant foods rich in phytochemicals.

A gene test recognizing the Thr/Met or Met/Met genotype of DDAH 1 gene is useful in selecting drug therapy for patients who might be at increased risk for adverse effects of nitric oxide antagonists or other compounds that inhibit the production of nitric oxide; either it will be possible to avoid the use of NO-antagonists in such patients, or it will be possible to include a specific NO-agonist in their therapeutic regimen.

The sequence of the nucleic acid can be used for screening a subject to determine if said subject is a carrier of a variant gene. The determination can be carried out either as a DNA analysis according to well known methods, which include direct DNA sequencing of the normal and variant gene, allele specific amplification using the polymerase chain reaction (PCR) enabling detection of either normal or variant sequence, or by indirect detection of the normal or variant gene by various molecular biology methods including e.g. PCR-single stranded conformation polymorphism (SSCP) method or denaturing gradient gel electrophoresis (DGGE). Determination of the normal or variant gene can also be done by using a restriction fragment length polymorphism (RFLP) method. Similarly, a test based on gene chip or array technology can be easily developed in analogy with many currently existing tests for single-nucleotide polymorphisms.

The determination can also be carried out at the level of RNA by analyzing RNA expressed at tissue level using various methods. Allele specific probes can be designed for hybridization. Hybridization can be done e.g. using Northern blot, RNase protection assay or in situ hybridization methods. RNA derived from the normal or variant gene can also be analyzed by converting tissue RNA first to cDNA and thereafter amplifying cDNA by an allele specific PCR method.

The kit for use in the method according to the invention preferably contains the various components needed for carrying out the method packaged in separate containers and/or vials and including instructions for carrying out the method. Thus, for example, some or all of the various reagents and other ingredients needed for carrying out the determination, such as buffers, primers, enzymes, control samples or standards etc can be packaged separately but provided for use in the same kit. Instructions for carrying out the method can be included inside the kit, as a separate insert, or as a label on the kit and/or on the separate vials. The kit may also contain the necessary software needed to interpret the results obtained with the kit, or for utilizing the results from a gene chip used in the method.

Based on the observations to be presented below NO availability or concentration enhancing therapies would be useful for treating a human or animal suffering from diseases in which abnormal NO metabolism is implicated. These include development of atherosclerosis and various pathological conditions, which predispose to atherosclerosis, such as hypercholesterolemia, hypertension, type 2 diabetes, hyperhomocyst (e) inemia and chronic renal failure (Lüscher TF et al 1993, WO 00/44888). Th e role of NO in type 2 (non-insulin dep endent) diabetes has been implcated earlier, even NO modifying treatments have been proposed for the treatment of diabetes. However, DDAH modifying treatments are a new approach in improving endothelial function among people suffering of diabetes mellitus. Abnormalities in endothelium-dependent vascular responses have been found among patients with insulin resistant diabetes mellitus (McVeigh G E et al 1992; Williams S B et al 1996). On the basis of our findings presented below, a defect in the DDAH I gene and thus a reduced activity of the DDAH protein, leading to retarded catabolism of ADMA and thus inhibition of nitric oxide, is associated with significantly elevation in the incidence of type 2 (non-insulin dependent) diabetes. On this basis we propose these treatments for type 2 diabetes and for its vascular complications.

Influence of the variant gene sequence can be investigated in transgenic animals. A transgenic animal can be generated e.g. using targeted homologous recombination methodology. This will provide an ideal preclinical model to investigate and screen new drug molecules, which are designed to modify the influence of the variant gene.

The invention will be described in more detail in the experimental section.

Experimental Section

Determination of Genomic Alleles Encoding the DDAH Gene

The method according to the invention for the determination of the allelic pattern of the codon in question can be carried out with polymerase chain reaction (PCR) in combination with either sequencing or restriction fragment length polymorphism methods.

Polymerase Chain Reaction (PCR)

The nucleotide sequence of the primer pair for the amplification of human dimethylarginine dimethylaminohydrolase 1 (DDAH1) exon number 1 was as follow: 5′-GTC CCC CGC CTC CGC ATA CTT-3′ (SEQ ID NO:5) and 5′-CCA CCT GCC CGA GAC CGT ACA A-3′ (SEQ ID NO:6). The size of the amplified DDAH1 exon 1 PCR product was 892 bp. The primers were designed by Mia Pirskanen and they were delivered by the DNA Synthesis and Sequencing Facility at the A.I. Virtanen Institute for Molecular Sciences (Kuopio, Finland).

The PCR amplification was conducted in a 20 μl volume: the reaction mixture contained 60 ng human genomic DNA (extracted from peripheral blood), 1× GeneAmp® Gold Buffer (Applied Biosystems, Foster City, Calif.), 1.25 mM of MgCl₂ (Applied Biosystems, Foster City, Calif.), 100 μM of each of the nucleotides (dATP, dCTP, dGTP, dTTP), 0.5 μM of each of the primers, 1 unit of the AmpliTaq Gold® DNA-polymerase (Applied Biosystems, Foster City, Calif.) and 1 M of Betaine (Sigma-Aldrich, Inc., Saint Louis, Mo., USA).

The target DNA sequence (exon 1 of the DDAH1 gene) was amplified in the above mentioned PCR reaction by using the GeneAmp® PCR System 9700 programmable thermo block (Applied Biosystems, Foster City, Calif.) with the PCR program conditions as follows: first the reaction was hold 7 minutes at 94° C., then the following two steps were repeated for 40 cycles: 45 seconds at 94° C., 1 minute and 30 seconds at 68° C., after which the reaction was kept at 72° C. for 5 minutes, and finally hold at 4° C.

Sequencing

Before the sequencing reaction the amplified DDAH1 gene exon 1 PCR product was purified with the GFX TM96 PCR Purification Kit (Amersham Pharmacia Biotech Inc, Piscataway, N.J.). The sequencing reaction was made by using the BigDye™ Terminator Cycle Sequencing v2.0 Ready Reactions with AmpliTaq® DNA Polymerase, FS DNA Sequencing Kit (Applied Biosystems, Foster City, Calif.). The sequencing primer for the DDAH1 exon number 1 was: 5′-CCG ACG GGA AGT TGT GAA-3 (SEQ ID NO:7). The sequencing primer was designed by Mia Pirskanen and it was delivered by the DNA Synthesis and Sequencing Facility at the A.I. Virtanen Institute for Molecular Sciences (Kuopio, Finland).

Cycle sequencing was made in the GeneAmp® PCR System 9700 programmable thermo block (Applied Biosystems, Foster City, Calif.) with the program as follows: the following three steps were repeated for 25 cycles; 10 seconds at 96° C., 5 seconds at 50° C. and 4 minutes at 60° C. after which the reaction hold at 4° C. (to perform cycle sequencing under standard conditions refer to ABI PRISM® 3100 Genetic Analyzer Sequencing Chemistry Guide, Applied Biosystems, Foster City, Calif.).

Dye terminator removal and sequencing reaction clean up was made using MultiScreen®-HV filtration plate (Millipore, Bedford, Mass.). After the purification the samples were transferred to MicroAmp® Optical 96-Well Reaction Plate (Applied Biosystems, Foster City, Calif.) and sequenced by using the ABI PRISM® 3100 Genetic Analyzer (Applied Biosystems, Foster City, Calif.), which is an automated fluorescence-based capillary electrophoresis DNA analysis system with 16 capillaries.

Restriction Fragment Length Polymorphism Method

The 892 bp exon 1 PCR-product of the DDAH1 gene was digested for 6 hours with BsmA I restriction endonuclease in the concentration of 1× NEBuffer 3 (New England BioLabs Inc., Beverly, Mass.), mixed with 6× loading dye solution and run in 1.7% agarose gel electrophoresis. Identification of normal and mutant alleles was based on different size of the restriction fragments in electrophoresis, resulting in distinct bands (normal homozygote form of Thr87 (Thr/Thr); 486 bp, 284 bp, 112 bp, 10 bp, heterozygote form of mutation Thr87Met (Thr/Met); 770 bp, 486 bp, 287 bp, 112 bp, 10 bp and homozygote form of mutation Thr87Met (Met/Met); 770 bp, 112 bp, 10 bp).

Population Study

The above referred population study of 1608 Finnish middle-aged male subjects including 13 subjects who were carriers of Thr/Met mutation of the DDAH gene is described in more detail in the following:

Knowing the vasoconstrictive property of ADMA through the competion with NO and the role of DDAH in the catabolism of ADMA, we hypothesized that the observed allelic variation in DDAH 1 gene would be associated with cardiovascular diseases such as coronary heart disease, cerebrovascular disease and hypertension as well as with diabetes. To test this hypothesis, we carried out a population study in 1608 middle-aged Finnish men. The study was carried out as part of the Kuopio Ischemic Heart Disease Risk Factor Study (KIHD), which is an ongoing population-based study designed to investigate risk factors for cardiovascular diseases, type 2 diabetes and related outcomes in men from eastern Finland (Salonen 1988). This area is known for its homogenous population (Sajantila et al. 1996), high coronary morbidity and mortality rates (Keys 1980) and a high incidence and prevalence of diabetes (Jousilahti Petal 1998).

Of the 1608 subjects, 13 (0.8%) were heterozygous carriers of the Thr/Met mutation of DDAH 1 gene. No homozygous Met/Met mutation carriers were found.

Of the 1595 non-carriers of the DDAH mutation, 320 (20.1%) had a prevalent coronary heart disease (CHD), whereas of the 13 carriers, nine (69.2%) had prevalent CHD. The unadjusted odds ratio is 9.0 (95% confidence interval (CI) 2.7 to 29.3, p<0.001). Prevalent CHD was defined as either a history of myocardial infarction or angina pectoris, angina pectoris on effort based on the London School of Hygiene questionnaire or regular use of nitroglycerin tablets. In a multivariate logistic model adjusting for age, examination years and smoking status, the carriers of the mutation had 9.4-fold risk of CHD (95% CI 2.4 to 36.8, p=0.001). In a step-up model including age, examination years, smoking status, body-mass index (kg/m2), serum selenium concentration, energy-adjusted dietary vitamin C intake and the ratio of dietary polyunsaturated to saturated fatty acids, of the mutation carriers had a 16.0-fold risk of CHD (95% CI 3.7 to 69.2, p<0.001).

Of the 1595 DDAH mutation non-carriers, 492 (30.8%) had CHD in exercise test, while of the 13 carriers, nine (69.2%) had exercise CHD. Exercise CHD was defined as either typical chest pain or ischaemic EKG changes during or after the exercise test (Kaplan and Salonen 1990, Laukkanen et al 2001). The unadjusted adds ratio was 5.0 (95% CI 1.5 to 16.5, p=0.005). In a logistic model adjusting for age, examination years and smoking status, this odds ratio was 6.0 (95% CI 1.5 to 23.1, p=0.010). In a step-up model adjusting for her strongest risk factors for exercise ischaemia, the odds ratio for the DDAH mutation carrier status was 6.8 (95% CI 1.7 to 26.9, p=0.006).

Nine strongest risk factors were entered simultaneously with the DDAH mutation carrier status in logistic models predicting prevalent CHD and CHD in exercise (Table 1). In these forced models, the mutation carriers had 9.6-fold risk of prevalent CHD and 5.3-fold risk of exercise-induced CHD.

The association of the DDAH mutation carrier status with prevalent hypertension was studied in the 1608 genotyped men. Hypertension was defined as systolic blood pressure (BP)≧165 mmHg or diastolic BP>95 nmihg or antihypertensive treatment. Both blood pressures were measured between 8:00 and 10:00 AM by one nurse with a random-zero mercury sphygmomanometer. The measuring protocol included three measurements in supine, one in standing and two in sitting position with 5-minutes intervals. The mean of all six measurements were used as systolic and diastolic blood pressures. In a logistic model adjusting for the strongest other risk factors for hypertension (age and body-mass index) and examination years, the DDAH mutation carriers had a 4.3-fold (95% CI 1.12 to 15.8, p=0.032) risk of hypertension, as compared with the non-carriers.

The association of the DDAH mutation carrier status with the incidence of diabetes was studied in 688 men who had no diabetes at baseline and who participated in a 11-year follow-up examination, in connection of which the diabetic status was re-examined. Diabetes was defined as fasting blood glucose concentration of 8.0 nmol/L or more or diagnosis and treatment for diabetes. In a logistic model adjusting for age, body-mass index, smoking status and examination years, the mutation carriers had a 6.9-fold risk of diabetes (95% CI 1.0 to 48.1, p=0.05). When another diabetes-causing mutation was added into the model, the odds ratio for the DDAH 1 mutation carrier status was 7.3 (95% CI 1.1 to 49.7, p=0.042).

The effect of the DDAH 1 genotype on the efficacy of nitroglycerin on chest pain, on angina pectoris was studied among the 1608 subjects. In men who were carriers of the DDAH 1 87^{th} met allele, the effect of the nitroglycerin on chest pain was less than among the non-carriers.

Futhermore, we studied the importance of ADMA in patho-biochemistry of diabetes related metabolic products in a set of 48 male subjects. Measurement of protein carbonyl groups (belongs to family of AGE) and ADMA were carried out at the baseline and 12 months later. The correlation co-efficient for association between ADMA and protein carbonyl groups was 0.48 (p<0.001) at entry and 0.46 (p=0.001) at 12 months. This is the first study to show a correlation between ADMA, a risk factor for endothelial dysfunction, and AGE, compounds that have been implicated in diabetes related complications.

Taken together, the known biological properties of ADMA and the DDAH, the homogeneity of the Finnish population, the study designs, the relatively large representative study population and the association of CHD, hypertension and diabetes with one trait suggest that the Thr/Met or Met/Met genotype of the DDAH 1 gene is a genetic risk factor for cardiovascular diseases and diabetes.

It will be appreciated that the methods of the present invention can be incorporated in the form of a variety of embodiments, only a few of which are disclosed herein. It will be apparent for the specialist in the field that other embodiments exist and do not depart from the spirit of the invention. Thus, the described embodiments are illustrative and should not be construed as restrictive.

TABLE 1
Relative probability of prevalent coronary heart disease (CHD) and CHD in exercise test and
its 95% confidence interval, related with DDAH 1 thr 87 met mutation carrier status and
other strongest risk factors in men. Results are from multivariate logistic regression models.

| | Prevalent CHD | | | CHD in exercise test | | |
|---|---|---|---|---|---|---|
| | Odds | | | Odds | | |
| Risk factor | ratio | 95% CI | P | ratio | 95% CI | P |
| DDAH 1 genotype (carrier vs. non-carrier) | 9.58 | 2.81, 32.7 | <0.001 | 5.26 | 1.56, 17.7 | 0.007 |
| Age (years) | 1.08 | 1.06, 1.11 | <0.001 | 1.07 | 1.04, 1.09 | <0.001 |
| Hair mercury content (μg/g) | 1.20 | 1.13, 1.28 | <0.001 | 1.13 | 1.07, 1.20 | <0.001 |
| Plasma fibrinogen concentration (g/L) | 1.47 | 1.15, 1.87 | 0.002 | 1.28 | 1.03, 1.07 | 0.023 |
| Hypertension years | 1.03 | 1.01, 1.05 | 0.005 | 1.05 | 1.03, 1.07 | <0.001 |
| Serum triglyceride concentration (mmol/L) | 1.25 | 1.07, 1.47 | 0.006 | 1.20 | 1.04, 1.40 | 0.014 |
| Dietary vitamin C intake (mg/d) | 0.997 | 0.99, 1.00 | 0.057 | 0.999 | 0.996, 1.000 | 0.274 |
| Serum apolipoprotein B concentration (g/L) | 1.39 | 0.76, 2.55 | 0.293 | 1.10 | 0.65, 1.85 | 0.735 |
| Serum HDL cholesterol concentration (mmol/L) | 0.86 | 0.53, 1.40 | 0.543 | 0.55 | 0.36, 0.84 | 0.006 |
| Cigarette-years of smoking | 1.00 | 1.000, 1.001 | 0.620 | 1.00 | 1.000, 1.000 | 0.652 |
| R square for the model | 0.159 | | | 0.129 | | |

### REFERENCES

Böger RHm Bode-Böger SM, Szuba A, et al. Asymmetric dimethylarginine (ADMA): a novel risk factor for endothelial dysfunction. Its role in hypercholesterolemia. Circulation 1998;98:1842-1847.

Fard A, Tuck C H, Donis J A, et al. Acute elevations of plasma asymmetric dimethylarginine and impaired endothelial function in response to a high-fat meal in patients with type 2 diabetes. Arterioscler Thromb Vasc Biol 2000;20:2039-2044.

Jousilahti P, Vartiainen E, Tuomilehto J, Pekkanen J, Puska P. Role of known risk factors in explaining the difference in the risk of coronary heart disease between eastern and southwestern Finland. Ann Med 1998;30(5):481-487.

Kaplan G A, Salonen J T. Socioeconomic conditions in childhood and ischaemic heart disease during middle age. BMJ 1990;301(6761):1121-1123.

Keys A: Seven Countries: A Multivariate Analysis of Death and Coronary Heart Disease. Cambridge, Mass, Harvard University Press, 1980.

Laukkanen J A, Kurl S, Lakka T A, et al. Exercise-induced silent myocardial ischemia and coronary morbidity and mortality in middle-aged men. J Am Coll Cardiol 2001;38(1):72-79.

Leiper J, MacAllister R, whitley G, Santa Maria J, Chubb A, Charles I, Vallance P. Identification of two human dimethylarginine dimethylaminohydrolases with distinct tissue distributions and homology to microbial arginine deiminases. Biochem J 1999;343:209-214.

Lüscher T F, Boulanger C M, Yang Z, Noll G, Dohi Y. Interactions between endothelium-derived relaxing and contracting factors in health and cardiovascular disease. Circulation 1993;87(suppl V):36-44

McVeigh G E, Brennan G M, Johnston G D, et al. Impaired endothelium-dependent and independent vasodilation in patients with type 2 (non-insulin-dependent) diabetes mellitus. Diabetologia 1992;35:771-776.

Miyazaki H, Matsuoka H, Cooke J P, et al. Endogenous nitric oxide synthase inhibitor: a novel marker of atherosclerosis. Circulation 1999;99:1141-1146.

Sajantila A, Salem A H, Savolainen P, Bauer K, Gierig C, Paabo S: Paternal and maternal DNA lineages reveal a bottleneck in the founding of the Finnish population. Proc Natl Acad Sci U.S.A. 1996;93:12035-12039.

Salonen J T. Is there a continuing need for longitudinal epidemiologic research? The Kuopio Ischaemic Heart Disease Risk Factor Study. Ann Clin Res 1988;20:46-50.

Tran Cam T L, Fox M F, Vallance P, Leiper J. DDAH 1 and DDAH 2 encode asymmetric methyl aminohydrolases with distinct expression patterns in adult and fetal tissues and are located on chromosomes 1p22 and 6p21.3 respectively. Genomics 2000;68:101-105.

Valkonen V P, Päivä H, Salonen J T, et al. Risk of acute coronary events and serum concentration of asymmetrical dimethylarginine. Lancet 2001 358(9299): 2127-2128.

Williams S B, Cusco J A, Roddy M A, Johnstone M T, Creager M A. Impaired nitric oxide-mediated vasodilation in patients with non-insulin-dependent diabetes mellitus. J Am Coll Cardiol 1996;27:567-574.

1. A nucleic acid encoding a variant DDAH 1 protein with a substitution of at least one of the amino acids selected from the group of 87^{th} to 94^{th} or 173^{rd} to 177^{th} or 221^{st} to 228^{th} amino acid residue of DDAH 1 protein.

2. The nucleic acid according to claim 1, wherein said nucleic acid is a genomic nucleotide sequence.

3. The nucleic acid according to claim 1 comprising a nucleotide sequence encoding a variant DDAH 1 protein with a substitution of threonine to metionine coded by codon 87 of the first exon of human DDAH 1 gene.

4. The nucleic acid according to claim 3 comprising the genomic nucleotide sequence of SEQ ID NO: 1.

5. The nucleic acid according to claim 3, wherein said nucleic acid is cDNA.

6. The nucleic acid according to claim 3 comprising a RNA sequence.

7. A variant DDAH 1 polypeptide having at least one substitution of the amino acids selected from the group of 87^{th} to 94^{th} or 173^{rd} to 177^{th} or 221^{st} to 228^{th} amino acid residue of DDAH 1 protein.

8. The variant DDAH 1 polypeptide according to claim 7 comprising the amino acid sequence of SEQ ID NO: 2.

9. A capturing probe which comprises a single strand of the cDNA according to claim 5.

10. A method for determining the presence or absence of a nucleic acid as defined in claim 1 in a biological sample, wherein said nucleic acid, as a target nucleic acid, appears in single stranded form and is brought into contact with a capturing nucleic acid probe and a detector nucleic acid probe, after which the complex of capturing probe, target nucleic acid and detector probe is detected.

11. The method according to claim 10, wherein the capturing nucleic acid probe is attached or capable of attaching to a solid phase, and comprises the cDNA sequence according to claim 5, and wherein a detected signal from the solid phase is an indication of the presence in the sample of a nucleic acid as defined in claim 1.

12. The method according to claim 11, wherein the capturing nucleic acid probe is attached or capable of attaching to a solid phase, and comprises a cDNA corresponding to the gene coding a wild-type DDAH 1 protein, and wherein a detected signal from the solid phase is an indication of the absence of the nucleic acid as defined in claim 1 in the sample.

13. A method for diagnosing a susceptibility to cardiovascular disease and diabetes in a subject by determining the pattern of alleles encoding a variant DDAH 1 protein, comprising the steps of
a) providing a biological sample of the subject to be tested,
b) detecting the presence of a variant genotype of the human DDAH I in the biological sample, the presence of the variant genotype indicating an increased risk of cardiovascular disease and diabetes in said subject.

14. The method according to claim 13, wherein said variant genotype of the human DDAH I is a heterozygote form of mutation Thr87Met (Thr/Met) or homozygote form of mutation Thr87Met (Met/Met).

15. The method according to claim 13, wherein the detection step is a DNA-assay.

16. The method according to claim 13, wherein the detection step is carried out using a gene or DNA chip, microarray, strip, panel or similar combination of more than one genes, mutations or RNA expressions to be assayed.

17. The method according to claim 13, wherein the allelic pattern is determined using polymerase chain reaction.

18. The method according to claim 13, wherein the biological sample is a blood sample or buccal swab sample and genomic DNA is isolated from said sample.

19. The method according to claim 13, wherein the detection step is based on a capturing probe, a single strand of cDNA, comprising a nucleotide sequence encoding a variant DDAH protein as defined in claim 7.

20. The method according to claim 13, wherein said method is used for determining whether a subject will benefit from treatment with a drug affecting the nitric oxide availability, production or metabolism of the subject.

21. The method according to claim 13, wherein said method is used for determining whether a subject will benefit from treatment with a drug reducing the ADMA availability or concentration of the subject.

22. The method according to claim 13, wherein said method is used for determining whether a subject will benefit from treatment with a DDAH availability or concentration elevating agent such as a DDAH agonist.

23. The method according to claim 13, wherein said method is used for determining whether a subject will be at increased risk of adverse effects if DDAH antagonists are administered to a subject.

24. The method according to claim 13, further comprising a step of selecting a subject with a DDAH gene sequence encoding a non-variant form of DDAH protein for clinical drug trials testing the antidiabetic, antihypertensive and myocardial ischaemia preventing effects of compounds.

25. A method for targeting the treatment of cardiovascular disease and diabetes in a hypertensive subject by determining the pattern of alleles encoding a a variant DDAH 1, i.e. by determining if said subject's genotype of the human DDAH 1 is of the variant type, comprising the steps presented in claim 13, and treating a subject of the variant genotype with a drug affecting the nitric oxide availability, production or metabolism of the subject.

26. The method according to claim 25, wherein said variant genotype of the human DDAH I is a heterozygote form of mutation Thr87Met (Thr/Met) or homozygote form of mutation Thr87Met (Met/Met).

27. The method according to claim 25, wherein said drug is a drug modulating, elevating or reducing the ADMA activity or concentration of the subjects either directly or through indirect such as central nervous system effects.

28. The method according to claim 25, wherein said drug is modulating, elevating or reducing the DDAH activity or concentration of the subjects either directly or through indirect such as central nervous system effects.

29. The method according to claim 25, wherein said drug is a subtype selective or nonselective DDAH agonist.

30. The method according to claims 25, wherein said cardiovascular disease is coronary heart disease, cerebrovascular disease or hypertension.

31. A method for treating a human or animal suffering from type 2 diabetes, said method comprising a therapy enhancing nitric oxide availability, production or concentration of the human subject or animal.

32. A method for treating vascular complications of diabetes, said method comprising a therapy enhancing nitric oxide availability, production or concentration of the human subject or animal.

33. The method according to claim 31, said method comprising administering to a subject a compound enhancing nitric oxide availability, production or concentration of the subject.

34. A method for treating a human or animal suffering from type 2 diabetes, said method comprising administering to a subject a compound reducing or inhibiting the ADMA activity or concentration of the subject either directly or through indirect such as central nervous system effects.

35. A method for treating a human or animal suffering from type 2 diabetes, said method comprising administering to a subject a compound elevating or inducing the DDAH activity or concentration of the subject either directly or through indirect such as central nervous system effects.

36. A method for treating a human or animal suffering from type 2 diabetes, said method comprising administering to a subject a compound inhibiting enzymes and other compounds that inhibit the formation of DDAH proteins and a compound activating activators of the formation of DDAH proteins.

37. The method according to claim 35, wherein said drug is a subtype (DDAH 1, DDAH 2) selective or nonselective DDAH agonist.

38. The method according to claims 31 and 32, wherein said therapy is gene therapy or gene transfer.

39. The method according to claim 38, wherein said therapy comprises the transfer of the non-variant DDAH 1 gene or fragment or derivative thereof.

40. A kit for detecting a risk of cardiovascular disease and diabetes in a subject, comprising means for determining the pattern of alleles encoding a variant DDAH in a biological sample from said subject, and optionally software to interpret the results of the determination.

41. A kit for determining the presence or absence of a nucleic acid as defined in claim 1 in a biological sample.

42. The kit according to claim 41 comprising a capturing nucleic acid probe.

43. A transgenic animal which carries a human DNA sequence comprising a nucleotide sequence encoding a variant DDAH 1 protein as defined in claim 7.
